(19) United States
(12) Patent Application Publication
(10) Pub. No.: US 2005/0124559 A1
(43) Pub. Date: Jun. 9, 2005

(54) Antimicrobial agent

(76) Inventors: Laurence Mark Von Itzstein, Palm Beach (AU); Ross Leon Coppel, Armadate (AU); Christopher Bonner Davis, Reedy Creek (AU); Robin Joy Thomson, Chevron Island (AU); David James Owen, BORONIA (AU)

Correspondence Address: MOORE & VAN ALLEN PLLC, P.O. BOX 13706, Research Triangle Park, NC, 27709, US

(21) Appl. No.: 10/505,119

(22) PCT Filed: Feb. 21, 2003
(86) PCT No.: PCT/AU03/00222

(30) Foreign Application Priority Data
Feb. 22, 2002 (AU) PS0691
Apr. 9, 2002 (AU) PS1623

(51) Int. Cl. A61K 31/70; C07H 5/04

(52) U.S. Cl. 514/24; 536/18.7

(57) ABSTRACT

A compound of general formula (I): 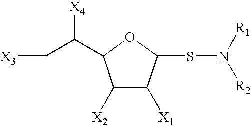
wherein R₁ and R₂ may be the same or different and are selected from the group consisting of hydrogen, optionally substituted C₄₋₃₀ alkyl and optionally substituted C₄₋₃₀ alkenyl, provided that R₁ and R₂ may not both be hydrogen, or R₁ and R₂ together with the nitrogen atom from which they depend form a saturated or unsaturated, optionally substituted heterocyclic group which may include additional heteroatoms selected from the group consisting of O, N and S, or R₁ and R₂ together with the nitrogen atom from which they depend form an optionally substituted lactam moiety;
X¹ is selected from the group consisting of OR₃, SR₃, NR₃R′₃, hydrogen, halogen, CN, C(O)NR₃R′₃, C(O)OR₃, OSO₃R₃, OPO₃R₃, NNR₃R′₃, SNR₃R′₃, NHSR₃, SSR₃ and substituted alkyl;
X₂ is selected from the group consisting of OR₄, SR₄, NR₄R′₄, hydrogen, halogen, CN, C(O)NR₄R′₄, C(O)OR₄, OSO₃R₄, OPO₃R₄, NNR₄R′₄, SNR₄R′₄, NHSR₄, SSR₄ and substituted alkyl;
X₃ is selected from the group consisting of OR₅, SR₅, NR₅R′₅, hydrogen, halogen, CN, C(O)NR₅R′₅, C(O)OR₅, OSO₃R₅, OPO₃R₅R′₅, NNR₅R′₅, SNR₅R′₅, NHSR₅, SSR₅ and substituted alkyl;
X₄ is selected from the group consisting of OR₆, SR₆, NR₆R′₆, hydrogen, halogen, CN, C(O)NR₆R′₆, C(O)OR₆, OSO₃R₆, OPO₃R₆R′₆, NNR₆R′₆, SNR₆R′₆, NHSR₆, SSR₆ and substituted alkyl;
R₃, R′₃, R₄, R′₄, R₅, R′₅, R₆ and R′₆ are the same or different and are selected from the group consisting of hydrogen, optionally substituted alkyl, optionally substituted aralkyl, optionally substituted aryl, optionally substituted acyl and a carbohydrate moiety; or a pharmaceutically acceptable salt thereof.

### TECHNICAL FIELD

The present invention relates to novel sulfenamides that have an antimicrobial action, methods for their synthesis, pharmaceutical compositions containing them and method of treatment of patients suffering microbial infection.

### BACKGROUND ART

Many bacterial diseases once thought to be on the decline are beginning to re-emerge and annually devastate populations in many countries. This problem is amplified by the emergence of many new drug resistant strains of the microorganisms that cause these diseases. Our interest in glycofuranose chemistry (Owen & von Itzstein, 2000) has led to the discovery of a new class of antimicrobial agents described below. Although significant chemistry and biology has been published (see, for example, Marino, Marino, Miletti, Alves, Colli, & de Lederkremer, 1998; Miletti, Marino, Marino, de Lederkremer, Colli & Alves, 1999; Zhang & Liu, 2001; Brimacombe, Gent & Stacey, 1968; Brimacombe, Da'aboul & Tucker, 1971; Lemieux & Stick, 1975; de Lederkremer, Cirelli & Sznaidman, 1986; Shin & Perlin, 1979; de Lederkremer, Cicero & Varela, 1990; de Lederkremer, Marino & Marino, 2002; Pathak, Pathak, Suling, Gurcha, Morehouse, Besra, Maddry & Reynolds, 2002; Ernst, Hart & Sinay, 2000) in the area of glycofuranose chemistry and biology none to date provides compounds that have significant antimicrobial activity.

### DISCLOSURE OF THE INVENTION

The present invention is concerned generally with novel sulfenamides that have antimicrobial action.

In a first aspect of the present invention there is provided a compound of general formula (I): 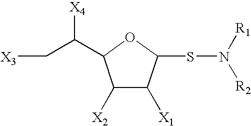
wherein R₁ and R₂ may be the same or different and are selected from the group consisting of hydrogen, optionally substituted C₄₋₃₀ alkyl and optionally substituted C₄₋₃₀ alkenyl, provided that R₁ and R₂ may not both be hydrogen, or R₁ and R₂ together with the nitrogen atom from which they depend form a saturated or unsaturated, optionally substituted heterocyclic group which may include additional heteroatoms selected from the group consisting of O, N and S, or R₁ and R₂ together with the nitrogen atom from which they depend form an optionally substituted lactam moiety;
X₁ is selected from the group consisting of OR₃, SR₃, NR₃R′₃, hydrogen, halogen, CN, C(O)NR₃R′₃, C(O)OR₃, OSO₃R₃, OPO₃R₃R₁₃, NNR₃R′₃, SNR₃R′₃, NHSR₃, SSR₃ and substituted alkyl;
X₂ is selected from the group consisting of OR₄, SR₄, NR₄R′₄, hydrogen, halogen, CN, C(O)NR₄R′₄, C(O)OR₄, OSO₃R₄, OPO₃R₄R₁₄, NNR₄R′₄, SNR₄R′₄, NHSR₄, SSR₄ and substituted alkyl;
X₃ is selected from the group consisting of OR₅, SR₅, NR₅R′₅, hydrogen, halogen, CN, C(O)NR₅R′₅, C(O)OR₅, OSO₃R₅, OPO₃R₅R′₅, NNR₅R′₅, SNR₅R′₅, NHSR₅, SSR₅ and substituted alkyl;
X₄ is selected from the group consisting of OR₆, SR₆, NR₆R₁₆, hydrogen, halogen, CN, C(O)NR₆R₁₆, C(O)OR₆, OSO₃R₆, OPO₃R₆R₁₆, NNR₆R₁₆, SNR₆R′₆, NHSR₆, SSR₆ and substituted alkyl;
R₃, R′₃, R₄, R′₄, R₅, R′₅, R₆ and R′₆ are the same or different and are selected from the group consisting of hydrogen, optionally substituted alkyl, optionally substituted aralkyl, optionally substituted aryl, optionally substituted acyl and a carbohydrate moiety;
or a pharmaceutically acceptable salt thereof.

The term “alkyl” used either alone or in a compound word such as “optionally substituted alkyl” or “optionally substituted cycloalkyl” denotes straight chain, branched or mono- or poly-cyclic alkyl. Examples of straight chain and branched C₄₋₃₀ alkyl include butyl, isobutyl, sec-butyl, tert-butyl, amyl, isoamyl, sec-amyl, 1,2-dimethylpropyl, 1,1-dimethylpropyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, nonyl, 1-, 2-, 3-, 4-, 5-, 6- or 7-methyloctyl, 1-, 2-, 3-, 4- or 5-ethylheptyl, 1-2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Examples of C₄₋₃₀ cycloalkyl include cyclobutyl, cyclopentyl, cyclohexyl, cycloheptyl, cyclooctyl, cyclononyl and cyclodecyl and the like.

The term “alkenyl” used either alone or in compound words such as “alkenyloxy” denotes groups formed from straight chain, branched or cyclic alkenes including ethylenically mono-, di- or poly-unsaturated alkyl or cycloalkyl groups as defined above. Examples of C₄₋₃₀ alkenyl include butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4,pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl.

The term “acyl” used either alone or in compound words such as “optionally substituted acyl” or “optionally substituted acyloxy” denotes an aliphatic acyl group or an acyl group containing an aromatic ring, which is referred to as aromatic acyl, or a heterocyclic ring, which is referred to as heterocyclic acyl, preferably C₁₋₃₀ acyl. Examples of acyl include straight chain or branched alkanoyl such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutyl, phenylpentanoyl and phenylhexanoyl) and naphthylalkanoyl (e.g. naphthylacetyl, naphthylpropanoyl and naphthylbutanoyl); aralkenoyl such as phenylalkenoyl (e.g. phenylpropenoyl, phenylbutenoyl, phenylmethacrylyl, phenylpentenoyl and phenylhexenoyl and naphthylalkenoyl (e.g. naphthylpropenoyl, naphthylbutenoyl and naphthylpentenoyl); heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; and heterocyclicalkenoyl such as heterocyclicpropenoyl, heterocyclicbutenoyl, heterocyclicpentenoyl and heterocyclichexenoyl.

The term “aryl” used either alone or in compound words such as “optionally substituted aryl”, “optionally substituted aryloxy” or “optionally substituted heteroaryl” denotes single, polynuclear, conjugated and fused residues of aromatic hydrocarbons or aromatic heterocyclic ring systems. Examples of aryl include phenyl, biphenyl, terphenyl, quaterphenyl, phenoxyphenyl, naphtyl, tetrahydronaphthyl, anthracenyl, dihydroanthracenyl, benzanthracenyl, dibenzanthracenyl, phenanthrenyl, fluorenyl, pyrenyl, indenyl, azulenyl, chrysenyl, pyridyl, 4-phenylpyridyl, 3-phenylpyridyl, thienyl, furyl, pyrryl, pyrrolyl, furanyl, imadazolyl, pyrrolydinyl, pyridinyl, piperidinyl, indolyl, pyridazinyl, pyrazolyl, pyrazinyl, thiazolyl, pyrimidinyl, quinolinyl, isoquinolinyl, benzofuranyl, benzothienyl, purinyl, quinazolinyl, phenazinyl, acridinyl, benzoxazolyl, benzothiazolyl and the like. Preferably, a carbocyclic aromatic ring system contains 6-10 carbon atoms and an aromatic heterocyclic ring system contains 1 to 4 heteratoms independently selected from N, O and S and up to 9 carbon atoms in the ring.

The term “heterocyclyl” or equivalent terms such as “heterocyclic” used either alone or in compound words such as “optionally substituted saturated or unsaturated heterocyclyl” denotes monocyclic or polycyclic heterocyclyl groups containing at least one heteroatom atom selected from nitrogen, sulphur and oxygen. Suitable heterocyclyl groups include N-containing heterocyclic groups, such as, unsaturated 3 to 6 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl or tetrazolyl;
saturated 3 to 6-membered heteromonocyclic groups containing 1 to 4 nitrogen atoms, such as, pyrrolidinyl, imidazolidinyl, piperidino or piperazinyl;
unsaturated condensed heterocyclic groups containing 1 to 5 nitrogen atoms, such as indolyl, isoindolyl, indolizinyl, benzimidazolyl, quinolyl, isoquinolyl, indazolyl, benzotriazolyl or tetrazolopyridazinyl;
unsaturated 3 to 6-membered heteromonocyclic group containing an oxygen atom, such as, pyranyl or furyl;
unsaturated 3 to 6-membered heteromonocyclic group containing 1 to 2 sulfur atoms, such as, thienyl;
unsaturated 3 to 6-membered heteromonocyclic group containing 1 to 2 oxygen atoms and 1 to 3 nitrogen atoms, such as, oxazolyl, isoxazolyl or oxadiazolyl;
saturated 3 to 6-membered heteromonocyclic group containing 1 to 2 oxygen atoms and 1 to 3 nitrogen atoms, such as, morpholinyl;
unsaturated condensed heterocyclic group containing 1 to 2 oxygen atoms and 1 to 3 nitrogen atoms, such as, benzoxazolyl or benzoxadiazolyl;
unsaturated 3 to 6-membered heteromonocyclic group containing 1 to 2 sulphur atoms and 1 to 3 nitrogen atoms, such as, thiazolyl or thiadiazolyl;
saturated 3 to 6-membered heteromonocyclic group containing 1 to 2 sulphur atoms and 1 to 3 nitrogen atoms, such as, thiazolidinyl; and
unsaturated condensed heterocyclic group containing 1 to 2 sulphur atoms and 1 to 3 nitrogen atoms, such as, benzothiazolyl or benzothiadiazolyl.

The term “carbohydrate” denotes a carbohydrate residue or a functionalised or deoxygenated carbohydrate residue, and includes monosaccharides and oligosaccharides. A carbohydrate residue is an acyclic polyhydroxy-aldehyde or ketone, or one of their cyclic tautomers. Oxygen atoms may be replaced by hydrogen or bonds to a halogen, nitrogen, sulfur or carbon atoms, or carbon-oxygen bonds such as in ethers or esters may be introduced. Examples of carbohydrates include but are not limited to D-galactofuranose, N-acetyl-D-galactofuranose, D-glucofuranose, N-acetyl-D-glucofuranose, D-galactopyranose N-acetyl-D-galactopyranose, D-glucopyranose and N-acetyl-D-glucopyranose and their equivalents where oxygen atoms have been replaced in selected positions with hydrogen or bonds to halogen, nitrogen, sulfur or carbon, as well as oligosaccharides containing these moieties.

In this specification “optionally substituted” means that a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyli, arylsulphenyl, carboalkoxy, carboaryloxy, mercapto, alkylthio, benzylthio, acylthio, phosphorus-containing groups and the like, provided that none of the substituents outlined above interferes with the formation of the subject compound.

Any of the moieties whose length is defined in terms of the number of carbon atoms present may possess any number of carbon atoms within the specified range. Nevertheless, within this range certain species will be preferred due to factors such as availability and cost of precursors and ease of synthesis, as well as efficacy. In particular, such moieties containing 4 to 24 carbon atoms, preferably 6 to 12 carbon atoms, more preferably 8 to 10 carbon atoms and most preferably 8 carbon atoms are preferred for reasons of cost and availability of precursors, ease of synthesis and efficacy.

In a particularly preferred embodiment of the present invention one of R₁ or R₂ is C₄₋₃₀ alkyl and the other is hydrogen or C₄₋₃₀ alkyl or R₁ and R₂ together with nitrogen atom from which they depend form a saturated or unsaturated heterocyclic ring containing said nitrogen atom as the single heteroatom.

More preferably, one of R₁ or R₂ is C₄₋₂₄, preferably C₆₋₁₂, alkyl and other is hydrogen or C₄₋₂₄, preferably, C₆₋₁₂, alkyl. More preferably still, one of R₁ or R₂ is C₈₋₁₀ alkyl and the other is hydrogen or C₈₋₁₀ alkyl. Advantageously, both R₁ and R₂ are C₄₋₃₀ alkyl, preferably C₄₋₂₄, more preferably C₆₋₁₂ alkyl and more preferably still C₈₋₁₀ alkyl, and most preferably C₈ alkyl. The alkyl groups are the same or different but most conveniently the same.

X₁, X₂, X₃ and X₄ may be any combination of substituents, but it is preferred that at least two of these moieties be other than hydrogen or a group linked to the ring through a carbon-carbon bond. Preferably, at least two of X₁, X₂, X₃ and X₄ are moieties linked to the ring through a carbon-oxygen bond, for example, in the case of X₁, OR₃, OSO₃R₃ and OPO₃R₃R′₃.

Preferably X₁ is OR₃. Advantageously R₃ is hydrogen or acyl, preferably C₁₋₃₀ acyl.

Preferably X₂ is OR₄. Advantageously R₄ is hydrogen or acyl, preferably C₁₋₃₀ acyl.

Preferably X₃ is OR₅. Advantageously R₅ is hydrogen or acyl, preferably C₁₋₃₀ acyl.

Preferably X₄ is OR₆. Advantageously R₆ is hydrogen or acyl preferably C₁₋₃₀ acyl.

Typically the compounds of the invention are galactofuranosyl compounds, and therefore have the configuration illustrated in general formula (Ia):

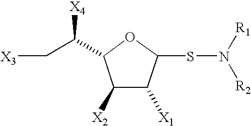

Alternatively, the compounds of the invention are glucofuranosyl derivatives having the general formula (Ib):

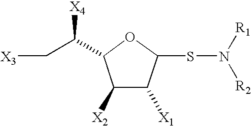

Advantageously the sulfenamide of general formula (I) is selected from the group consisting of N,N-Didecyl-S-(2,3,5,6-tetra-O-benzoyl-1-thio-β-D-galactofuranosyl)sulfenamide, N,N-Dioctyl-S-(2,3,5,6-tetra-O-benzoyl-1-thio-β-D-galactofuranosyl) sulfenamide, N,N-Dihexyl-S-(2,3,5,6-tetra-O-benzoyl-1-thio-β-D-galactofuranosyl)sulfenamide, N,N-Didecyl-S-(1-thio-β-D-galactofuranosyl)sulfenamide, N,N-Dioctyl-S-(1-thio-β-D-galactofuranosyl)sulfenamide, N,N-Dihexyl-S-(1-thio-β-D-galactofuranosyl)sulfenamide, N,N-Dioctyl-S-(2,3,5,6-tetra-O-acetyl-1-thio-β-D-glucofuranosyl)sulfenamide and N,N-Dioctyl-S— (1-thio-β-D-glucofuranosyl) sulfenamide.

In a particularly preferred embodiment of the invention the sulfenamide of general formula (I) is N,N-Didecyl-S-(1-thio-β-D-galactofuranosyl)sulfenamide, N,N-Dioctyl-S-(1-thio-β-D-galactofuranosyl)sulfenamide or N,N-Dihexyl-S-(1-thio-β-D-galactofuranosyl)sulfenamide, most particularly, N,N-Dioctyl-S-(1-thio-β-D-galactofuranosyl)sulfenamide.

According to a second aspect of the present invention there is provided a method of preparation of a compound of general formula (I): 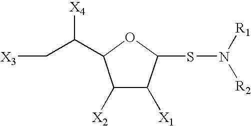
comprising reacting a compound of general formula (II): 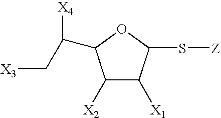
wherein Z is an acyl group, preferably acetyl and X₁, X₂, X₃ and X₄ are as defined above with the proviso that none of R₃, R′₃, R₄, R′₄, R₅, R′₅, R₆ and R′₆ is hydrogen but, instead, is a protecting group;
with a compound of general formula (III): 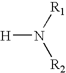
wherein R₁ and R₂ are as defined above;
in the presence of a bis-activated alkyl halide; and, optionally removing the protecting groups.

Typically the bis-activated alkyl halide is diethyl bromomalonate, trimethyl bromophosphonoacetate or N-bromosuccinimide. In general terms the reaction is performed in the presence of an excess of the secondary amine of general formula (III) in an inert solvent such as DMF or THF, or mixtures of such solvents, at a temperature from 20° C. to 60° C., preferably 25° C. to 40° C., under an atmosphere of nitrogen or argon. The reaction mixture may be left to stir typically for 2 to 160 hours, preferably greater than 24 hours, prior to isolation and purification, or deprotection. Suitable protecting groups are well known to the person skilled in the art and in this case the benzoyl group is preferred. Benzoyl protecting groups are typically removed through hydrolysis with sodium methoxide in methanol. The compounds of the present invention may also be synthesised through the condensation of sulfenyl halides with a secondary amine of general formula (III), the reaction of the relevant thiols and amines in the presence of oxidising reagents or via the reaction of disulfides and amines in the presence of silver or mercuric salts. An extensive array of methodologies has been developed to manipulate each position of the furanose template as disclosed, for example, in Marino, Marino, Miletti, Alves, Colli, & de Lederkremer, 1998; Miletti, Marino, Marino, de Lederkremer, Colli & Alves, 1999; Zhang & Liu, 2001; Brimacombe, Gent & Stacey, 1968; Brimacombe, Da'aboul & Tucker, 1971; Lemieux & Stick, 1975; de Lederkremer, Cirelli & Sznaidman, 1986; Shin & Perlin, 1979; de Lederkremer, Cicero & Varela, 1990; de Lederkremer, Marino & Marino, 2002; Pathak, Pathak, Suling, Gurcha, Morehouse, Besra, Maddry & Reynolds, 2002; Ernst, Hart & Sinay, 2000; the contents of which are incorporated herein by reference.

According to a third aspect of the present invention there is provided a method for the treatment of a patient with a microbial infection, comprising administering to said patient a therapeutically effective amount of a compound of general formula (I).

According to a fourth aspect of the present invention there is provided the use of a compound of general formula (I) in the manufacture of a medicament for use in the treatment of a microbial infection.

As used herein, the term “therapeutically effective amount” means an amount of a compound of the present invention effective to yield a desired therapeutic response, for example to prevent or treat a disease which by administration of a pharmaceutically-active agent.

The specific “therapeutically effective amount” will, obviously, vary with such factors as the particular condition being treated, the physical condition and clinical history of the subject, the type of animal being treated, the duration of the treatment, the nature of concurrent therapy (if any), and the specific formulations employed and the structure of the compound or its derivatives.

As used herein, a “pharmaceutical carrier” is a pharmaceutically acceptable solvent, suspending agent, excipient or vehicle for delivering the compound of general formula (I) to the subject. The carrier may be liquid or solid, and is selected with the planned manner of administration in mind.

The compound of general formula (I) may be administered orally, topically, or parenterally in dosage unit formulations containing conventional non-toxic pharmaceutically acceptable carriers, adjuvants, and vehicles. The term parenteral as used herein includes subcutaneous, intravenous, intramuscular, intrathecal, intracranial, injection or infusion techniques.

The invention also provides suitable topical, oral, aerosol, and parenteral pharmaceutical formulations for use in the novel methods of treatment of the present invention. The compounds of the invention may be administered orally as tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable preparations. The tablets contain the active ingredient in admixture with non-toxic pharmaceutically acceptable excipients which are suitable for the manufacture of tablets.

These excipients may be, for example, inert diluents, such as calcium carbonate, lactose, calcium phosphate or sodium phosphate; granulating and disintegrating agents, such as corn starch or alginic acid; binding agents, such as starch, gelatin or acacia; or lubricating agents, such as magnesium stearate, stearic acid or talc. The tablets may be uncoated, or may be coated by known techniques to delay disintegration and absorption in the gastrointestinal tract and thereby provide a sustained action over a longer period. For example, a time-delay material such as glyceryl monostearate or glyceryl distearate may be employed. Coating may also be performed using techniques described in the U.S. Pat. Nos. 4,256,108; 4,160,452; and 4,265,874 to form osmotic therapeutic tablets for control release.

The compound of general formula (I) of the invention can be administered, for in vivo application, parenterally by injection or by gradual perfusion over time independently or together. Administration may be intravenously, intra-arterial, intraperitoneally, intramuscularly, subcutaneously, intracavity, or transdermally. For in vitro studies the agents may be added or dissolved in an appropriate biologically acceptable buffer and added to a cell or tissue.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers such as those based on Ringer's dextrose, and the like. Preservatives and other additives may also be present such as, for example, anti-microbials, anti-oxidants, chelating agents, growth factors and inert gases and the like.

The compounds of general formula (I) are antimicrobial agents which are active, in particular but not limited to, against Mycobacterium including Mycobacterium tuberculosis, M. avium intracellulare, M. fortuitum, M. abscessus and rapid growing atypical Mycobacterial strains, Nocardia, particularly Nocardia asteroides and N. nova, Staphylococcus including Staphylococcus aureus and S. aureus (Coagulas-negative) and Enterococci species. The compounds of general formula (I) are particularly useful in treating infections involving these organisms.

Generally, the terms “treating”, “treatment” and the like are used herein to mean affecting a subject, tissue or cell to obtain a desired pharmacological and/or physiological effect. The effect may be prophylactic in terms of completely or partially preventing infection, and/or may be therapeutic in terms of a partial or complete cure of an infection. “Treating” as used herein covers any treatment of, or prevention of infection in a vertebrate, a mammal, particularly a human, and includes: preventing the infection from occurring in a subject that may have been exposed to the infectious agent, but has not yet been diagnosed as affected; inhibiting the infection, ie., arresting its development; or relieving or ameliorating the effects of the infection, ie., cause regression of the effects of the infection.

According to a fifth aspect of the present invention there is provided a pharmaceutical composition comprising a compound of general formula (I) and a pharmaceutically acceptable carrier.

The pharmaceutical compositions according to one embodiment of the invention are prepared by bringing a compound of general formula (I) into a form suitable for administration to a subject using carriers excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412,1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the microbial infection and the weight and general state of the subject. Typically, dosages used in vitro may provide useful guidance in the amounts useful for in situ administration of the pharmaceutical composition, and animal models may be used to determine effective dosages for treatment of the cytotoxic side effects. Various considerations are described, eg., in Langer, Science, 249: 1527, (1990). Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleaginous suspension. This suspension may be formulated according to known methods using suitable dispersing or wetting agents and suspending agents such as those mentioned above. The sterile injectable preparation may also a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

Compounds of general formula (I) may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Dosage levels of the compound of general formula (I) of the present invention will usually be of the order of about 0.05 mg to about 20 mg per kilogram body weight, with a preferred dosage range between about 0.05 mg to about 10 mg per kilogram body weight per day (from about 0.1 g to about 3 g per patient per day). The amount of active ingredient which may be combined with the carrier materials to produce a single dosage will vary, depending upon the host to be treated and the particular mode of administration. For example, a formulation intended for oral administration to humans may contain about 1 mg to 1 g of an active compound with an appropriate and convenient amount of carrier material, which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In addition, some of the compounds of the invention may form solvates with water or common organic solvents. Such solvates are encompassed within the scope of the invention.

The compounds of the invention may additionally be combined with other compounds to provide an operative combination. It is intended to include any chemically compatible combination of pharmaceutically-active agents, as long as the combination does not eliminate the activity of the compound of general formula (I) of this invention.

According to a sixth aspect of the present invention there is provided a method of killing a microorganism, comprising exposing said microorganism to a compound of general formula (I) as defined above.

Advantageously, although not limited to, the microorganism is selected from the group consisting of Mycobacterium including Mycobacterium tuberculosis, M. avium intracellulare, M. fortuitum, M. abscessus and rapid growing atypical Mycobacterial strains, Nocardia, particularly Nocardia asteroides and N. nova, Staphylococcus including Staphylococcus aureus and S. aureus (Coagulas-negative) and Enterococci species.

Throughout this specification and the claims, the words “comprise”, “comprises” and “comprising” are used in a non-exclusive sense, except where the context requires otherwise.

It will be clearly understood that, although a number of prior art publications are referred to herein, this reference does not constitute an admission that any of these documents forms part of the common general knowledge in the art, in Australia or in any other country.

Modes for Performing the Invention

The synthetic scheme employed to prepare compounds in accordance with preferred embodiments of the invention is now described in more detail. For the preparation of Examples 1 to 6, per-O-benzoylated GalfSAc (compound 2) was prepared according to known literature methods (Owen & von Itzstein, 2000) and is shown in Scheme 1 without modification. All new compounds gave the expected spectroscopic data. The synthesis of protected (compound 3; Examples 1, 2 and 3) and deprotected (compound 1; Examples 4, 5 and 6) galactofuranosyl N,N-dialkylsulfenamides is described in Scheme 1. 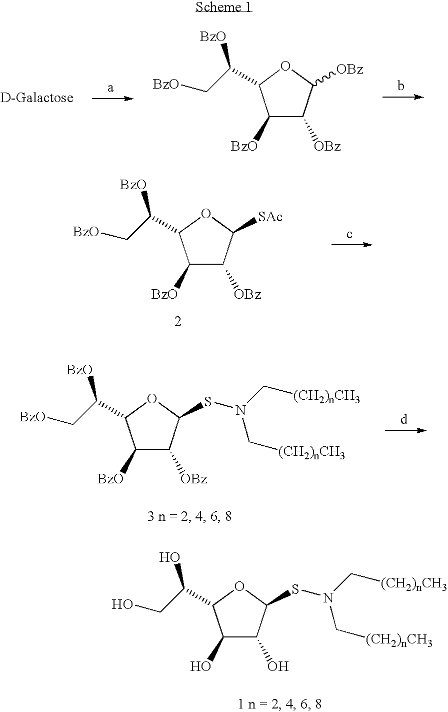
Reagents and Conditions: a) i) pyr, 100° C., 1 h, ii) BzCl, 60° C., 2 h, iii) rt, 24 h; b) SnCl₄, CH₂Cl₂, HSAc, rt, 1 h, N₂; c) BrCH(COOEt)₂, DMF, THF, HN[CH₂(CH₂)ₙCH₃]₂, rt/40° C., 12-80 h, N₂; d) NaOMe, MeOH, rt, 2 h, N₂.

For the preparation of Examples 7 and 8, per-O-acetylated GlcfoAc (compound 4) was prepared according to known literature methods (Furneaux, Rendle and Sims, 2000) and is shown in Scheme 2 without modification. All new compounds gave the expected spectroscopic data. The synthesis of protected (compound 6; Example 7) and deprotected (compound 7; Example 8) glucofuranosyl N,N-dialkylsulfenamides is described in Scheme 2. 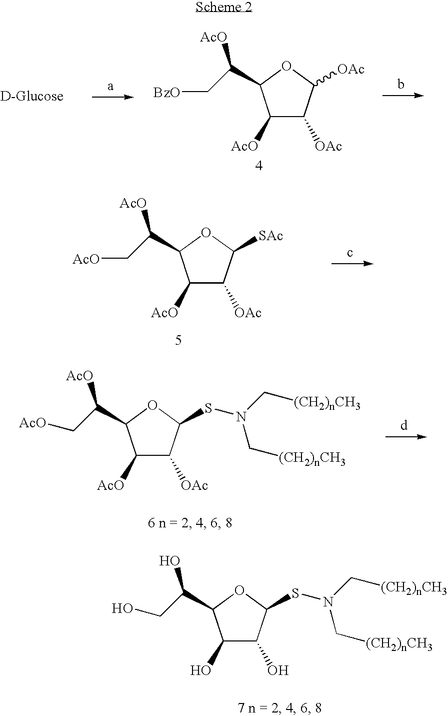
Reagents and Conditions: a) i) H₃BO₃, CH₃CO₂H, 50° C., 1 h, ii) (CH₃CO)₂O, 50° C., 16 h, iii) MeOH, iv) (CH₃CO)₂O, pyr, 25° C., 2 h; b) SnCl₄, CH₂Cl₂, HSAc, rt, 1.5 h, N₂; c) BrCH(COOEt)₂, DMF, THF HN[CH₂(CH₂)ₙCH₃]₂, rt/40° C., 140 h, N₂; d) NaOMe, MeOH, rt, 1 h, N₂.

### Example 1

### N,N-Didecyl-S-(2,3,5,6-tetra-O-benzoyl-1-thio-β-D-galactofuranosyl)sulfenamide 3 (n=8)

The didecylamine (in an 8-10 fold excess compared with the thioacetate 2) was dissolved in a 1:1 mixture of dry DMF/THF (generally 60 mL) and heated to 40° C. under an atmosphere of N₂. To this solution was added in one portion, a mixture of 1-S-acetyl-2,3,5,6-tetra-O-benzoyl-1-thio-β-D-galactofuranose 2 (0.5 mmol) and diethyl bromomalonate (1-2 mmol, 2-4 equiv) dissolved in a 1:1 mixture of dry DMF/THF (4 mL). The reaction was left to stir at room temperature overnight. The next morning all volatile compounds were carefully removed under high vacuum with gentle heating (to approximately 50° C.). The resultant amorphous solid residue was dissolved in boiling ethyl acetate and then left to cool to room temperature where upon the excess amine crystallized. The solid was removed by filtration and the solvent removed from the mother liquors under reduced pressure. The resulting orange residue was purified by flash chromatography. 35% yield. R_{f} 0.6 (hexane/EtOAc, 5:1). ¹H NMR (300 MHz, CDCl₃): δ 7.26-8.10 (m, 20H, 4×COC₆H₅), 6.05 (m, 1H, H-5), 5.78 (d, 1H, J_{1,2} 2.8 Hz, H-1), 5.67 (dd, 1H, J_{3,2} 2.1, J_{3,4} 4.9 Hz, H-3), 5.53 (dd, 1H, J_{2,3} 2.1, J_{2,1} 2.8 Hz, H-2), 4.80 (app.t, 1H, J 4.5, H-4), 4.74 (m, 2H, H-6 and H-6′), 2.95 (m, 4H, 2×NCH₂), 1.58 (t, 4H, J 6.6 Hz, didecyl chain), 1.23 (br.s, 28H, didecyl chain), 0.87 (t, 6H, J 6.7 Hz, didecyl chain); ¹³C NMR (75.5 MHz, CDCl₃): □ 166.0, 165.7, 165.6, 165.3 (4×CO₂C₆H₅), 133.5, 133.3, 133.2, 133.1, 130.0, 129.8, 129.7, 129.6, 129.6, 129.1, 128.5, 128.4, 128.3, (aromatic C), 98.8 (C-1), 81.3 (C-4), 79.9 (C-2), 78.2 (C-3), 70.7 (C-5), 63.4 (C-6), 58.6 (2×NCH₂), 31.9, 29.7, 29.6, 29.5, 29.3, 28.5, 26.9, 22.7 (16×CH₂, didecyl chain), 14.1 (2×CH₃, didecyl chain); HRMS calcd for C₅₄H₆₉NO₉S.H⁺ 908.47713, Found 908.47653.

### Example 2

### N,N-Dioctyl-S-(2,3,5,6-tetra-O-benzoyl-1-thio-β-D-galactofuranosyl)sulfenamide 3 (n=6)

1-S-Acetyl-2,3,5,6-tetra-O-benzoyl-1-thio-β-D-galactofuranose 2 (780 mg, 1.19 mmol) was dissolved in dry THF (25 mL). Diethylbromomalonate (305 μL, 1.79 mmol, 1.5 molar equiv.) was then added, and the mixture was stirred for 10 minutes at room temperature under N₂. Dioctylamine (1.44 mL, 4.76 mmol, 4 molar equiv.) was then added and the reaction stirred for 70 h at room temperature under Argon. After this time the volatile compounds were removed under reduced pressure. The residue was then diluted in EtOAc (100 mL) and washed twice with sat. NaCl (2×100 mL), dried over Na₂SO₄, filtered, and the solvent removed under reduced pressure. The residue was chromatographed (hexane-EtOAc 16:1, then hexane-EtOAc 6:1. TLC; R_{f} 0.57, hexane-EtOAc 4:1) to furnish N,N-dioctyl-S-(2,3,5,6-tetra-O-benzoyl-1-thio-β-D-galactofuranosyl) sulfenamide as a pale orange syrup. (601 mg, 59%). R_{f} 0.57 (hexane-EtOAc 4:1). ¹H NMR (300 MHz, CDCl₃): δ 7.26-8.11 (m, 20H, 4×CO₂Ph), 6.06 (m, 1H, H-5), 5.77 (d, 1H, J 3.0 Hz, H-1), 5.66 (dd, 1H, J_{3,2} 2.1, J_{3,4} 5.0 Hz, H-3), 5.52 (dd, 1H, J_{2,3} 2.3, J_{2,1} 2.9-Hz, H-2), 4.79 (app t, 1H, J 4.5 Hz, H-4), 4.73 (m, 2H, H-6 and H-6′), 2.94 (m, 4H, 2×NCH₂), 1.56 (m, 4H, 2×CH₂, dioctyl chain), 1.25 (m, 20H, 10×CH₂, dioctyl chain), 0.85 (t, 6H, J 6.6 Hz, 2×CH₃); ¹³C NMR (75.5 MHz, CDCl₃): δ 166.0, 165.7, 165.6, 165.3 (4×CO₂Ph), 133.5, 133.3, 133.2, 133.0, 133.0, 129.8, 129.7, 129.6, 129.5, 129.0, 128.5, 128.4, 128.3 (CO₂Ph), 90.8 (C-1), 81.3 (C-4), 79.9 (C-2), 78.2 (C-3), 70.7 (C-5), 63.4 (C-6), 58.6 (2×NCH₂), 31.8, 29.5, 29.3, 28.4, 26.9, 22.6 (12×CH₂, dioctyl chain), 14.2 (2×CH₃); LRMS (ESI): m/z 875 [(M+Na)⁺ 45%] 471 (93) 227 (100).

### Example 3

### N,N-Dihexyl-S-(2,3,5,6-tetra-O-benzoyl-1-thio-β-D-galactofuranosyl)sulfenamide 3 (n=4)

1-S-Acetyl-2,3,5,6-tetra-O-benzoyl-1-thio-β-D-galactofuranose 2 (193 mg, 0.3 mmol) was dissolved in dry DMF (3 mL). Dry THF (3 mL) was then added followed by diethylbromomalonate (453 μL, 9 molar equiv.) and the mixture was allowed to stir for 10 minutes at room temperature. Dihexylamine (1.0 mL, approx. 15 molar excess) was then added and the reaction allowed to stir for 40 h at room temperature under N₂. After this time the volatile compounds were removed under reduced pressure with heating to 35° C. for 24 h. The waxy residue was then diluted in EtOAc (100 mL) and the hydrobromide salt of the excess amine crystallised out and was filtered from solution. The EtOAc solution was washed twice with brine (2×100 mL), dried over Na₂SO₄, filtered, and the solvent removed under reduced pressure. The residue was chromatographed twice (silica, #1 hexane-EtOAc 8:1; #2 hexane-EtOAc 16:1. TLC; R_{f} 0.54 hexane-EtOAc 6:1) to furnish N,N-dihexyl-S-(2,3,5,6-tetra-O-benzoyl-1-thio-β-D-galactofuranosyl) sulfenamide as a pale orange syrup. (50 mg, 21%). R_{f} 0.54 (hexane/EtOAc 6:1). ¹H NMR (300 MHz, CDCl₃): δ 7.26-8.12 (m, 20H, 4×CO₂C₆H₅), 6.06 (m, 1H, H-5), 5.77 (d, 1H, J_{1,2} 3.1 Hz, H-1), 5.67 (dd, 1H, J_{3,2} 2.1 Hz, J_{3,4} 5.0 Hz, H-3), 5.52 (dd, 1H, J_{2,3} 2.3 Hz, J_{2,1} 3.0 Hz, H-2), 4.79 (t, 1H, J 4.5 Hz, H-4), 4.73 (m, 2H, H-6 and H-6′), 2.95 (m, 4H, 2×NCH₂), 1.57 (m, 4H, dihexyl chain), 1.24 (m, 12H, dihexyl chain), 0.84 (t, 6H, J 6.6 Hz, 2×CH₃); ¹³C NMR (75.5 MHz, CDCl₃): □ 166.2, 165.9, 165.8, 165.6 (4×CO₂Ph), 133.7, 133.5, 133.4, 133.3, 130.2, 130.2, 130.0, 129.9, 128.7, 128.6, 128.5 (CO₂Ph), 91.0 (C-1), 81.5 (C-4), 80.1 (C-2), 78.4 (C-3), 70.9 (C-5), 63.4 (C-6), 58.8 (2×NCH₂), 31.9, 28.6, 26.7, 22.8 (8×CH₂, dihexyl chain), 14.2 (2×CH₃); LRMS (ESI) m/z 818 [(M+Na)⁺ 38%] 796 (32) 186 (100).

General Procedure for the Deprotection of Benzoate Protecting Groups:

To a solution of the protected sulfenamide (0.5 mmol) in dry methanol (10 mL) under an atmosphere of N₂ was added one equivalent of sodium methoxide (1 M solution in dry methanol). The reaction was left to sir at room temperature for 2 h. After this time the reaction was neutralized with Amberlite (H⁺) resin. The resin was removed by filtration and the solvent removed under reduced pressure to yield the desired deprotected compound.

### Example 4

N,N-Didecyl-S-(1-thio-β-D-galactofuranosyl)sulfenamide 1 (n=8): Yield: 0.50%. R_{f} 0.4 (EtOAc). ¹H NMR (300 MHz, CD₃OD): δ 5.20 (d, 1H, J_{1,2} 5.4 Hz, H-1), 4.07 (dd, 1H, J_{3,2} 5.6 Hz, J_{3,4} 7.7 Hz, H-3), 3.86 (dd, 1H, J_{4,5} 2.4 Hz, J_{4,3} 7.7 Hz, H-4), 3.76-3.72 (m, 2H, H-2 and H-5), 3.62 (m, 2H, H-6 and H-6′), 2.92 (m, 4H, 2×NCH₂), 1.60 (t, 4H, J 6.4 Hz, didecyl chain), 1.30 (br s, 28H, didecyl chain), 0.90 (t, 6H, J 6.5 Hz, didecyl chain); ¹³C NMR (75.5 MHz, CD₃OD): □ 92.4 (C-1), 82.8 (C-4), 80.7 (C-2), 78.0 (C-3), 72.1 (C-5), 65.2 (C-6), 59.8 (2×NCH₂), 33.2, 31.0, 30.9, 30.9, 30.8, 30.7, 30.6, 29.5, 28.6, 23.9 (16×CH₂, didecyl chain), 14.6 (2×CH₃, didecyl chain); LRMS (ESI) m/z 492 [(M+H)⁺ 20%] 298 (100); HRMS calcd for C₂₆H₅₃NO₅S.H⁺ 492.37227, Found 492.37368.

### Example 5

N,N-Dioctyl-S-(1-thio-β-D-galactofuranosyl)sulfenamide 1 (n=6): Yield: 46%. R_{f} 0.29 (EtOAc). ¹H NMR (300 MHz, CD₃OD): δ 5.16 (d, 1H, J 5.5 Hz, H-1), 4.02 (dd, 1H, J_{3,2} 5.6, J_{3,4} 7.8 Hz, H-3), 3.81 (dd, 1H, J_{4,3} 7.8, J_{4,5} 2.5 Hz, H-4), 3.72 (app t, 1H, J 5.5 Hz, H-2), 3.68 (dd, 1H, J_{5,4} 2.5, J_{5,6} 6.3 Hz, H-5), 3.57 (m, 2H, H-6 and H-6′), 2.88 (m, 4H, 2×NCH₂), 1.56 (m, 4H, 2×CH₂, dioctyl chain), 1.27 (m, 20H, 10×CH₂, dioctyl chain), 0.86 (t, 6H, J 6.6 Hz, 2×CH₃); ¹³C NMR (75.5 MHz, CD₃OD): □ 92.2 (C-1), 82.6 (C-4), 80.5 (C-2), 77.8 (C-3), 71.9 (C-5), 65.0 (C-6), 59.6 (2×NCH₂), 33.1, 30.6, 30.5, 29.4, 27.9, 23.7 (12×CH₂, dioctyl chain), 14.5 (2×CH₃); LRMS (ESI) m/z 458 [(M+Na)⁺ 9%] 436 (7) 242 (100).

### Example 6

N,N-Dihexyl-S-(1-thio-β-D-galactofuranosyl)sulfenamide 1 (n=4): Yield: 62%. R_{f} 0.52 (EtOAc/MeOH 7:2). ¹H NMR (300 MHz, CD₃OD): δ 5.17 (d, 1H, J_{1,2} 5.5 Hz, H-1), 4.03 (dd, 1H, J_{3,2} 5.6 Hz, J_{3,4} 7.8 Hz, H-3), 3.83 (dd, 1H, J_{4,3} 7.8 Hz, J_{4,5} 2.5 Hz, H-4), 3.73 (app.t, 1H, J 5.5 Hz, H-2), 3.70 (dd, 1H, J_{5,4} 2.5 Hz, J_{5,6} 6.3 Hz, H-5), 3.58 (m, 2H, H-6 and H-6′), 2.89 (m, 4H, 2×NCH₂), 1.57 (quintet, 4H, J 6.9 Hz, dihexyl chain), 1.27 (m, 12H, dihexyl chain), 0.87 (t, 6H, J 6.8 Hz, 2×CH₃); LRMS (ESI) m/z 402 [(M+Na)⁺ 6%] 242 (5) 186 (100).

### Example 7

### 1-S-Acetyl-2,3,5,6-tetra-O-acetyl-1-thio-β-D-glucofuranose 5

To a stirred solution of 1,2,3,5,6-penta-O-acetyl-β-D-glucofuranose 4 (1.99 g, 5.1 mmol) in dry CH₂Cl₂ (20 mL) at 0° C., under N₂ was added tin tetrachloride (660 μL, 5.6 mmol). After 10 minutes thiolacetic acid (730 μL, 10.3 mmol) was added and the reaction was stirred for 80 minutes at 0° C. under N₂. After this time the reaction was diluted with sat. aq. NaHCO₃ (150 mL) and EtOAc (150 mL). The layers were separated and the organic layer was washed once with sat. aq. NaHCO₃ (150 mL) and once with aq. NaCl (150 mL). The organic phase was then dried over Na₂SO₄, filtered, and the solvent removed under reduced pressure. The residue was chromatographed (hexane-EtOAc 2:1, TLC; R_{f} 0.38, Hex-EtOAc 2:1) to furnish 1-S-acetyl-2,3,5,6-tetra-O-acetyl-1-thio-β-D-glucofuranose as a thick clear syrup (2.07 g, 72%). R_{f} 0.38 (Hex-EtOAc 2:1). ¹H NMR (300 MHz, CDCl₃): δ 5.91 (s, 1H, H-1), 5.39 (d, 1H, J_{3,4} 3.8 Hz, H-3), 5.23 (ddd, 1H, J_{5,6} 2.4, J_{5,6} 4.9, J_{5,4} 9.5 Hz, H-5), 5.17 (s, 1H, H-2), 4.57 (dd, 1H, J_{6,6′} 12.3, J_{6,5} 2.4 Hz, H-6), 4.43 (dd, 1H, J_{4,5} 9.5, J_{4,3} 3.8 Hz, H-4), 4.10 (m, 1H, H-6′), 2.38 (s, 3H, SCOCH₃), 2.00-2.14 (4×s, 12H, OCOCH₃); ¹³C NMR (75.5 MHz, CDCl₃): δ 192.7 (SCOCH₃), 170.5, 169.6, 169.0 (4×OCOCH₃), 85.4 (C-1), 80.9 (C-2), 79.2 (C-4), 73.3 (C-3), 67.9 (C-5), 63.0 (C-6), 30.7 (SCOCH₃), 20.7, 20.7, 20.7, 20.6 (4×OCOCH₃); LRMS (ESI): m/z 429 [(M+Na)⁺ 100%].

N,N-Dioctyl-S— (2,3,5,6-tetra-O-acetyl-1-thio-β-D-glucofuranosyl) sulfenamide 6 (n=6).

To a solution of 1-S-acetyl-2,3,5,6-tetra-O-acetyl-1-thio-β-D-glucofuranose 5 (375 mg, 0.92 mmol) in dry THF (20 mL) was added diethylbromomalonate (314 μL, 1.84 mmol, 2 molar equiv.) and the mixture was stirred for 10 minutes at room temperature under Ar. Dioctylamine (1.12 mL, 3.69 mmol, 4 molar equiv.) was then added and the reaction was stirred for 140 h at room temperature under Ar. After this time the volatile compounds were removed under reduced pressure. The residue was then diluted in EtOAc (100 mL) and the solution washed twice with aq. NaCl (2×100 mL), dried over Na₂SO₄, filtered, and the solvent removed under reduced pressure. The residue was chromatographed (hexane-EtOAc 16:1, then hexane-EtOAc 4:1. TLC; R_{f} 0.74, hexane-EtOAc 2:1) to furnish N,N-dioctyl-S— (2,3,5,6-tetra-O-acetyl-1-thio-β-D-glucofuranosyl) sulfenamide as a pale yellow syrup (175 mg, 26%). R_{f} 0.74 (hexane-EtOAc 2:1). ¹H NMR (300 MHz, CDCl₃): δ 5.32 (dd, 1H, J_{3,4} 4.1, J_{3,2} 1.2 Hz, H-3), 5.27 (ddd, 1H, J_{5,6} 2.4, J_{5,6′} 4.9, J_{5,4} 9.3 Hz, H-5), 5.18 (d, 1H, J_{1,2} 2.7 Hz, H-1), 5.08 (dd, 1H, J_{2,3} 1.2, J_{2,1} 2.7 Hz, H-2), 4.59 (dd, 1H, J_{6,6′} 12.3, J_{6,5} 2.4 Hz, H-6), 4.31 (dd, 1H, J_{4,5} 9.3, J_{4,3} 4.1 Hz, H-4), 4.16 (dd, 1H, J_{6′,6} 12.3, J_{6′,5} 4.9 Hz, H-6′), 2.87 (broad t, 4H, 2×NCH₂), 2.00-2.12 (3×s, 12H, 4×OCOCH₃), 1.50-1.62 (m, 4H, 2×CH₂), 1.20-1.36 (m, 20H, 10×CH₂), 0.88 (t, 6H, J 6.7 Hz, 2×CH₃); ¹³C NMR (75.5 MHz, CDCl₃): δ 170.6, 169.7, 169.4, 169.3 (4×OCOCH₃), 91.2 (C-1), 79.7 (C-2), 78.4 (C-4), 74.1 (C-3), 68.2 (C-5), 63.0 (C-6), 58.7 (2×NCH₂), 31.9, 29.5, 29.3, 28.3, 26.9, 22.7 (12×CH₂), 20.8, 20.7 (4×OCOCH₃), 14.1 (2×CH₃); LRMS (ESI) m/627 [(M+Na)⁺ 67%] 642 (32) 605 (22) 242 (100).

### Example 8

### N,N-Dioctyl-S-(1-thio-β-D-glucofuranosyl) sulfenamide 7 (n=6)

To a solution of N,N-dioctyl-S-(2,3,5,6-tetra-O-acetyl-1-thio-β-D-glucofuranosyl) sulfenamide (140 mg, 0.23 mmol) in dry MeOH (10 ml) was added one equivalent of NaOMe (230 μL, 1M solution in dry MeOH). The reaction was stirred for 70 minutes at room temperature under N₂. After this time the solution was neutralised with Amberlite IR 120 (H⁺) resin, filtered, and the solvent removed under reduced pressure. The residue was chromatographed (EtOAc. TLC; R_{f} 0.29, EtOAc-hexane 4:1) to yield N,N-dioctyl-S-(1-thio-β-D-glucofuranosyl) sulfenamide as a white amorphous solid (45 mg, 45%). R_{f} 0.29 (EtOAc-hexane 4:1). ¹H NMR (300 MHz, CD₃OD): δ 4.92 (d, 1H, J 2.6 Hz, H-1), 3.98 (m, 2H, H-2 and H-3), 3.87 (m, 2H, H-4 and H-5), 3.72 (m, 1H, H-6), 3.55 (dd, 1H, J_{6′,6} 16.8, J_{6′,5} 5.5 Hz, H-6′), 2.82 (m, 4H, 2×NCH₂), 1.47-1.61 (m, 4H, 2×CH₂), 1.16-1.33 (m, 20H, 10×CH₂), 0.83 (app t, J 6.5 Hz, J 6.9 Hz, 2×CH₃); LRMS (ESI) m/z 458 [(M+Na)⁺ 24%] 436 (8) 242 (100).

Biological Data

### Example 9

Inhibition of various bacteria by compound 1 (n=8) is described in Table 1. The biological data were determined by either the BACTEC or Mueller-Hinton agar method and expressed as a minimum inhibitory concentration (MIC).

TABLE 1

| Genus | Susceptibility | MIC | |
|---|---|---|---|
| Mycobacterium tuberculosis | + | <5 | μg/ml |
| M. avium intracellulare | + | <5 | μg/ml |
| M. fortuitum | + | <5 | μg/ml |
| M. abscessus | + | <5 | μg/ml |
| Rapid Growing Atypical | + | <5 | μg/ml |
| Mycobacterial Strains | | | |
| Nocardia asteroides | + | <5 | μg/ml |
| N. nova | + | <5 | μg/ml |
| Staphylococcus aureus | + | 5-50 | μg/ml |
| S. aureus (Coagulas- | + | 5-50 | μg/ml |
| negative) | | | |
| Enterococci sp. | + | 5-50 | μg/ml |

### Example 10

Inhibition of various bacteria by compound 1 (n=6, and n=4) are described in Table 2. The biological data were determined by a Zone Inhibition Assay method. The zone of inhibition was measured using an arbitrary scale: (+++=large zone of inhibition, −=no zone of inhibition). Compound 1 (n=6, and n=4) were tested by spotting 3 μL of a 10 μg/mL sample onto a filter disc which was placed on a lawn of bacteria. N.B. Compound 1 (n=8) was used as a positive control.

| | | Zone of |
|---|---|---|
| Organism tested | Compound | Inhibition |
| Mycobacterium smegmatis | 1 (n = 4) | ++ |
| | 1 (n = 6) | +++ |
| Bacillus subtilis | 1 (n = 4) | ++ |
| | 1 (n = 6) | +++ |
| Staphylococcus aureus | 1 (n = 4) | + |
| | 1 (n = 6) | +++ |
| Enterococcus faecalis | 1 (n = 4) | + |
| | 1 (n = 6) | +++ |

### Industrial Applicability

The compounds of general formula (I) are anti-microbial agents.

### REFERENCES

The disclosure of the following documents is incorporated herein by reference:
Owen, D. J.; von Itzstein, M. Carbohyr. Res. 2000, 328, 287-292.
Marino, C.; Marino, K.; Miletti, L. C.; Alves, M. J. M.; Colli, W.; de Lederkremer, R. M. Glycobiology 1998, 8, 901-904.
Miletti, L. C.; Marino, C.; Marino, K.; de Lederkremer, R. M.; Colli, W.; Alves, M. J. M. Carbohydr. Res. 1999, 320, 176-182.
Zhang, Q.; Liu, H. J. Am. Chem. Soc. 2001, 123, 6756-6766.
Brimacombe, J.; Gent, P.; Stacey, M. J. Chem. Soc. Org. 1968, 567-569.
Brimacombe, J.; Da'aboul, I.; Tucker, L. J. Chem. Soc. Org. 1971, 3762.
Lemieux, R. U.; Stick, R. V. Aust. J. Chem. 1975, 28, 1799-1801.
de Lederkremer R. M.; Cirelli, A.; Sznaidman, M. L. Carbohydr. Res. 1986, 146, 233-240.
Shin, J.; Perlin, A. Carbohydr. Res. 1979, 76, 165-176.
de Lederkremer R. M.; Cicero, D.; Varela, O. Tetrahedron 1990, 46, 1131-1144.
de Lederkremer, R. M.; Marino, K.; Marino, C. Anal. Biochem. 2002, 301, 325-328.
Pathak, A. K.; Pathak, V.; Suling, W. J.; Gurcha, S. S.; Morehouse, C. B.; Besra, G. S.; Maddry, J. A.; Reynolds, R. C. Bioorg. Med. Chem. 2002, 10, 923-928.
Ernst, B.; Hart, G. W.; Sinay, P. (Eds), Carbohydrates in Chemistry and Biology, Wiley-VCH, Weinheim, 2000, Vols 1-4.
Furneaux, R. H.; Rendle, P. M.; Sims, I. M. J. Chem. Soc. Perkin Trans 1 2000, 13, 2010-2014.

1. A compound of general formula (I): 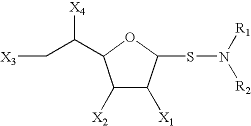
wherein R₁ and R₂ may be the same or different and are selected from the group consisting of hydrogen, optionally substituted C₄₋₃₀ alkyl and optionally substituted C₄₋₃₀ alkenyl, provided that R₁ and R₂ may not both be hydrogen, or R₁ and R₂ together with the nitrogen atom from which they depend form a saturated or unsaturated, optionally substituted heterocyclic group which may include additional heteroatoms selected from the group consisting of O, N and S, or R₁ and R₂ together with the nitrogen atom from which they depend form an optionally substituted lactam moiety;
X₁ is selected from the group consisting of OR₃, SR₃, NR₃R′₃, hydrogen, halogen, CN, C(O)NR₃R′₃, C(O)OR₃, OSO₃R₃, OPO₃R₃R₁₃, NNR₃R′₃, SNR₃R′₃, NHSR₃, SSR₃ and substituted alkyl;
X₂ is selected from the group consisting of OR₄, SR₄, NR₄R′₄, hydrogen, halogen, CN, C(O)NR₄R′₄, C(O)OR₄, OSO₃R₄, OPO₃R₄R₁₄, NNR₄R′₄, SNR₄R′₄, NHSR₄, SSR₄ and substituted alkyl;
X₃ is selected from the group consisting of OR₅, SR₅, NR₅R₁₅ hydrogen, halogen, CN, C(O)NR₅R′₅, C(O)OR₅, OSO₃R₅, OPO₃R₅R₁₅, NNR₅R′₅, SNR₅R′₅, NHSR₅, SSR₅ and substituted alkyl;
X₄ is selected from the group consisting of OR₆, SR₆, NR₆R′₆ hydrogen, halogen, CN, C(O)NR₆R′₆, C(O)OR₆, OSO₃R₆, OPO₃R₆R₁₆, NNR₆R₁₆, SNR₆R′₆, NHSR₆, SSR₆ and substituted alkyl; R₃, R′₃, R₄, R′₄, R₅, R′₅, R₆ and R′₆ are the same or different and are selected from the group consisting of hydrogen, alkyl, substituted alkyl, aralkyl, substituted aralkyl, aryl, substituted aryl, acyl, substituted acyl and a carbohydrate moiety;
or a pharmaceutically acceptable salt thereof.

2. A compound as claimed in claim 1 wherein one of R₁ or R₂ is C₄₋₂₄ alkyl and the other is hydrogen or C₄₋₂₄ alkyl.

3. A compound as claimed in claim 2 wherein one of R₁ or R₂ is C₆₋₁₂ alkyl and the other is hydrogen or C₆₋₁₂ alkyl.

4. A compound as claimed in claim 3 wherein one of R₁ or R₂ is C₈₋₁₀ alkyl and the other is hydrogen or C₈₋₁₀ alkyl.

5. A compound as claimed in claim 1 wherein both R₁ or R₂ are C₄₋₃₀ alkyl.

6. A compound as claimed in claim 1 wherein X₁ is OR₃.

7. A compound as claimed in claim 6 wherein R₃ is hydrogen or C₁₋₃₀ acyl.

8. A compound as claimed in claim 1 wherein X₂ is OR₄.

9. A compound as claimed in claim 8 wherein R₄ is hydrogen or C₁₋₃₀ acyl.

10. A compound as claimed in claim 1 wherein X₃ is OR₅.

11. A compound as claimed in claim 10 wherein R₅ is hydrogen or C₁₋₃₀ acyl.

12. A compound as claimed in claim 1 wherein X₄ is OR₆.

13. A compound as claimed in claim 12 wherein R₆ is hydrogen or C₁₋₃₀ acyl.

14. A compound as claimed in claim 1 having the general formula (Ia):

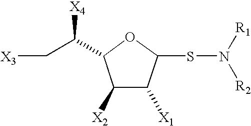

15. A compound as claimed in claim 1 having the general formula (Ib):

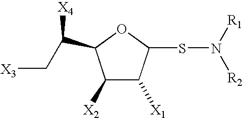

16. A compound selected from the group consisting of:
N,N-Didecyl-S-(2,3,5,6-tetra-O-benzoyl-1-thio-β-D-galactofuranosyl)sulfenamide
N,N-Dioctyl-S-(2,3,5,6-tetra-O-benzoyl-1-thio-β-D-galactofuranosyl)sulfenamide
N,N-Dihexyl-S-(2,3,5,6-tetra-O-benzoyl-1-thio-β-D-galactofuranosyl)sulfenamide
N,N-Didecyl-S-(1-thio-β-D-galactofuranosyl)sulfenamide
N,N-Dioctyl-S-(1-thio-β-D-galactofuranosyl)sulfenamide
N,N-Dihexyl-S-(1-thio-β-D-galactofuranosyl)sulfenamide
N,N-Dioctyl-S-(2,3,5,6-tetra-O-acetyl-1-thio-β-D-glucofuranosyl)sulfenamide and
N,N-Dioctyl-S-(1-thio-β-D-glucofuranosyl)sulfenamide.

17. A method of preparation of a compound of general formula (I): 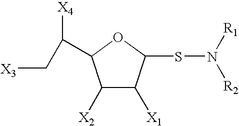
comprising reacting a compound of general formula (II): 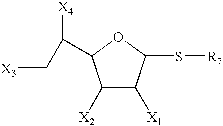
wherein is an acyl group, preferably acetyl and X₁, X₂, X₃ and X₄ are as defined above with the proviso that none of R₃, R′₃, R₄, R′₄, R₅, R′₅, R₆ and R′₆ is hydrogen but, instead, is a protecting group;
with a compound of general formula (III): 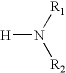
wherein R₁ and R₂ as defined above;
in the presence of a bis-activated alkyl halide.

18. A method for the treatment of a patient with a microbial infection, comprising administering to said patient a therapeutically effective amount of a compound of general formula (I) as claimed in claim 1.

19. (canceled)

20. A pharmaceutical composition comprising a compound of general formula (I) as claimed in claim 1 and a pharmaceutically acceptable carrier.

21. A method of killing a microorganism, comprising exposing said microorganism to a compound of general formula (I) as claimed in claim 1.

22. A method as claimed in claim 17, further including removing the protecting groups.
